(19) United States
(12) Patent Application Publication
(10) Pub. No.: US 2021/0052231 A1
(43) Pub. Date: Feb. 25, 2021

(54) METHOD AND SYSTEM FOR ANALYZING RISK ASSOCIATED WITH RESPIRATORY SOUNDS

(71) Applicant: SALCIT TECHNOLOGIES PRIVATE LIMITED, Bachupally (IN)

(72) Inventors: Narayana Rao Venkata Naga Sripada, Hyderabad (IN); Manmohan Jain, Hyderabad (IN); ShubhaDeepti Palreddy, Hyderabad (IN); Srinivas Mukund Vadrev, Hyderabad (IN); Venkataramaiah Yechuri, Visakhapatnam (IN); Vamsi Priya Yechuri, Visakhapatnam (IN); Vardhan Seshasai Avasarala, Mason, OH (US)

(73) Assignee: SALCIT TECHNOLOGIES PRIVATE LIMITED, Bachupally (IN)

(21) Appl. No.: 17/081,149

(22) Filed: Oct. 27, 2020

Related U.S. Application Data
(63) Continuation-in-part of application No. 16/768,872, filed on Jun. 1, 2020.

(30) Foreign Application Priority Data
Dec. 14, 2017 (IN) 201741045066

(51) Int. Cl. A61B 5/00; A61B 5/08; A61B 7/00; A61B 5/01; A61B 5/145; G16H 10/60; G16H 50/30; G16H 50/20; G16H 50/70; G16H 50/50; G06Q 10/10; G16H 40/67; G10L 25/66

(52) U.S. Cl. CPC A61B 5/7275; A61B 2562/06; A61B 7/003; A61B 5/01; A61B 5/14542; A61B 5/7267; A61B 5/746; A61B 5/742; A61B 5/0002; G16H 10/60; G16H 50/30; G16H 50/20; G16H 50/70; G16H 50/50; G06Q 10/105; G16H 40/67; G10L 25/66; A61B 2503/20; A61B 5/7475; A61B 5/6898; A61B 5/0823

(57) ABSTRACT

Disclosed herein is a method and system for monitoring health risk of a user. The system receives a plurality of information related to clinical symptoms and cough sounds of a user and determines a symptom risk index and a cough risk index based on analysis of clinical symptoms and cough sounds. The system further receives body temperature and oxygen saturation information of the user and determines temperature risk index and oxygen saturation risk index based on the analysis of the received body temperature and oxygen saturation information. The system further determines a final risk index of the user based on the determined symptom risk index, cough risk index, temperature risk index and oxygen saturation risk index. The system provides a quick and easy solution to estimate or predict health risk information of the user.

### CROSS REFERENCE TO RELATED APPLICATIONS

This application is a continuation-in-part to U.S. Application No. 16/768,872 filed on 1 Jun. 2020 which is the U.S. national phase of International patent application No. PCT/IN2018/050745 filed 13 Nov. 2018.

### FIELD OF THE DISCLOSURE

Embodiments of the present disclosure are related, in general to health advisory, and more particularly, but not exclusively to a method and system for monitoring health risk associated with a person and provide health insights to the person.

### BACKGROUND

Throughout the human history, the mankind has experienced a number of epidemics and pandemics of infectious diseases such as smallpox, tuberculosis, HIV/AIDS, COVID 19 etc., wherein such diseases kill thousands of people and also spread infection in the society. The infectious diseases represent a considerable threat to the society in spite of significant medical progress over last centuries. Such infectious diseases further risk the health of people performing various operations in one or more enterprises, organizations, government offices, health care centres, schools etc. Employee health affects everything from workplace culture, absenteeism, performance, and productivity, to health and safety in the workplace. Governmental and non-governmental organizations face a huge challenge to manage the risk of bringing employees to office during such pandemic or epidemic. Therefore, as a preventive measure, such organizations deploy a plurality of solutions to determine any risk in employees' health and take necessary decision based on determined risk.

Conventional solutions for determining risk in employees' health include guided set of questionnaires answered by the employees, body temperature reading of plurality of employees etc. However, the existing solutions do not provide a complete risk analysis of a person as the existing solutions do not consider monitoring of one or more important health parameters such as oxygen saturation, cough characteristics, blood components etc. Therefore, there is a need for a method and system to monitor health risk of a person based on combined analysis of guided questionnaire, body temperature, oxygen saturation information, cough sound and other health parameters and determine risk assessment of group of people of any organization continually over the time to provide health insights of the person or the group of people.

The information disclosed in this background of the disclosure section is only for enhancement of understanding of the general background of the disclosure and should not be taken as an acknowledgement or any form of suggestion that this information forms prior art already known to a person skilled in the art.

### SUMMARY

One or more shortcomings of the prior art are overcome, and additional advantages are provided through the present disclosure. Additional features and advantages are realized through the techniques of the present disclosure. Other embodiments and aspects of the disclosure are described in detail herein and are considered a part of the claimed disclosure.

Accordingly, the present disclosure relates to a method of monitoring and assessing health risk of a user. The method includes receiving a plurality of information related to clinical symptoms and cough sounds from the user. The method comprises determining a symptom risk index based on analysis of the received plurality of clinical symptoms data and computing a cough risk index based on one or more cough sound characteristics as determined by processing the received plurality of cough sounds. The method further comprises determining a temperature risk index and an oxygen saturation risk index based on analysis of measured body temperature reading and oxygen saturation level of the user. The method comprises generating a final risk index based on the computed symptom risk index, cough risk index, temperature risk index, and oxygen saturation risk index. The method further comprises automatically assessing the health risk of the user as one of safe-to-work and not safe-to-work based on the final risk index.

Further, the disclosure relates to a system for monitoring and assessing health risk of a user. The system comprises a processor, and a memory communicatively coupled with the processor, wherein the memory stores processor-executable instructions, which, on execution, cause the processor to receive a plurality of information related to clinical symptoms and cough sounds from the user. The processor is configured to determine a symptom risk index based on analysis of the received plurality of clinical symptoms data and compute a cough risk index based on one or more cough sound characteristics as determined by processing the received plurality of cough sounds. The processor is configured to determine a temperature risk index and an oxygen saturation risk index based on analysis of measured body temperature reading and oxygen saturation level of the user. The processor generates a final risk index based on the computed symptom risk index, cough risk index, temperature risk index, and oxygen saturation risk index. The processor further automatically assesses the health risk of the user as one of safe-to-work and not safe-to-work based on the final risk index.

The foregoing summary is illustrative only and is not intended to be in anyway limiting. In addition to the illustrative aspects, embodiments, and features described above, further aspects, embodiments, and features will become apparent by reference to the drawings and the following detailed description.

### BRIEF DESCRIPTION OF DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of device or system and/or methods in accordance with embodiments of the present subject matter are now described, by way of example only, and with reference to the accompanying figures, in which:

FIG. 1 illustrates an exemplary architecture of a proposed system for monitoring health risk of a user in accordance with some embodiments of the present disclosure;

FIG. 2 illustrates an exemplary block diagram of a health monitoring system (HMS) in accordance with an embodiment of the present disclosure;

FIG. 3 illustrates an exemplary block diagram of two-step process for health risk assessment in accordance with some embodiments of the present disclosure;

FIG. 4 illustrates an exemplary representation for training of a machine learning model of the HMS in accordance with some embodiments of the present disclosure;

FIG. 5A illustrates a flowchart of a method for monitoring health risk of a user in accordance with some embodiments of the present disclosure;

FIG. 5B illustrates a flowchart of a method for determining cough risk index of FIG. 4A in accordance with some embodiments of the present disclosure;

FIG. 5C illustrates a flowchart of a process for generating alert upon determination of risk in the health of the user in accordance with some embodiments of the present disclosure;

FIG. 6A-6B illustrate a cough severity scoring table and a final risk index scoring table respectively in accordance with some embodiments of the present disclosure;

FIG. 6C-6D illustrate exemplary graphical representations for cough count trends for dry cough and wet cough respectively in accordance with some embodiments of the present disclosure; and

FIG. 7 illustrates a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a device or system or apparatus proceeded by “comprises . . . a” does not, without more constraints, preclude the existence of other elements or additional elements in the device or system or apparatus.

Embodiments of the present disclosure relates to a method and system for monitoring and assessing health risk of a user. In one embodiment, the system receives a plurality of information related to clinical symptoms and cough sounds from the user. The system determines a symptom risk index based on analysis of the received information related to clinical symptoms. The system determines one or more cough sound characteristics by processing the received cough sounds using a machine learning model and computes a cough risk index based on the analysis of determined one or more cough sound characteristics. The system further determines a temperature risk index and an oxygen saturation risk index based on analysis of measured body temperature reading and oxygen saturation level of the user. The system generates a final risk index based on the computed symptom risk index, cough risk index, temperature risk index, and oxygen saturation risk index. The system automatically assesses the health risk of the user as one of safe-to-work and not safe-to-work based on the final risk index, generates an alert when the health risk of the user is not safe-to-work and transmits the generated alert to the user and one or more authorized persons related to the user.

The system monitors cough sound characteristics, body temperature, oxygen saturation level of the user for a period of time and determines an individual threshold value of the user for each of the monitored health parameter. Upon determining individual threshold values, the system determines a trend in the values of the user's health parameters i.e. body temperature, oxygen saturation level, cough sounds based on captured health parameters' information, individual threshold values, and the set of corresponding pre-defined threshold values. The system further provides health insights of the user by predicting future health risk conditions based on the analysis of the determined trend. The ability of the system to analyse the plurality of health parameters and determine the final risk index based on the analysis of plurality of health parameters of the user, results in a quick and easy solution to decision making in a risky health condition and predict health risk of the user.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

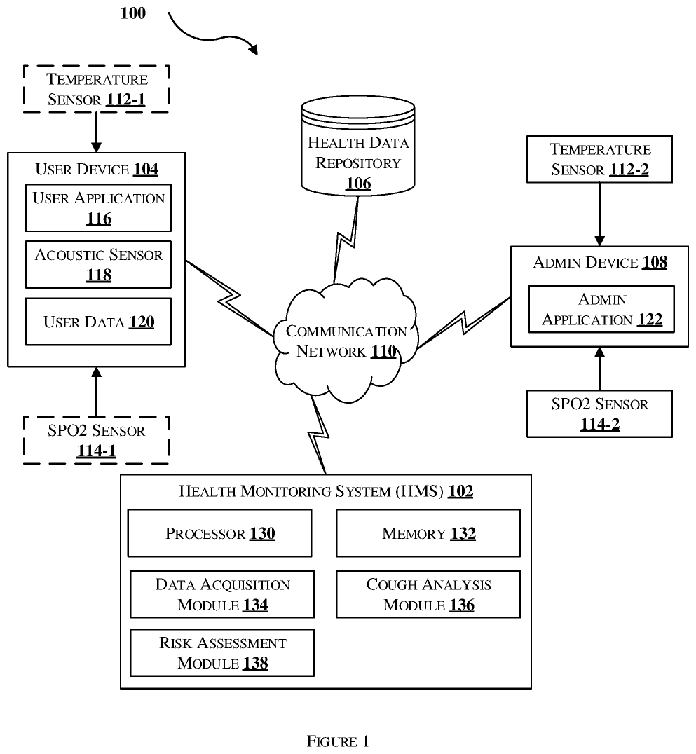

FIG. 1 illustrates an exemplary architecture of a proposed system to monitor health risk of a user in accordance with some embodiments of the present disclosure.

As shown in FIG. 1, the exemplary system (100) comprises one or more components configured for monitoring and assessing health risk of a user in real time. In one embodiment, the system (100) comprises a health monitoring system (hereinafter referred to as HMS) (102), a user device (104), a health data repository (106), and an admin device (108) communicatively coupled via a communication network (110). The user device (104) may be communicatively coupled to a temperature sensor (112-1), and a SPO2 (Peripheral Capillary Oxygen Saturation) sensor (114-1). The admin device (108) may also be further communicatively coupled to a temperature sensor (112-2), and a SPO2 (Peripheral Capillary Oxygen Saturation) sensor (114-2). The communication network (110) may include, without limitation, a direct interconnection, LAN (local area network), WAN (wide area network), wireless network, point-to-point network, or another configuration. One of the most common types of network in current use is a TCP/IP (Transfer Control Protocol and Internet Protocol) network for communication between database client and database server. Other common Internet protocols used for such communication include HTTPS, FTP, AFS, and WAP and other secure communication protocols etc.

The health data repository (106) is capable of storing plurality of operational data of HMS (102) in the respective dataset. The health data repository (106) is configured to store the plurality of information such as user information, and user's health related information like temperature data, oxygen saturation information, cough sounds, clinical symptoms data etc. recorded on regular intervals. The health data repository (106) is further capable of storing the plurality of user's health related information recorded over a period of time such as for week, month etc., for trend analysis and prediction of future health risk. Further, the health data repository (106) also stores a set of training datasets to train a machine learning model for determining cough severity for the received plurality of cough sounds based on the training. In one embodiment, the health data repository (106) may be integrated within the HMS (102). In another embodiment, the health data repository (106) may be configured as a standalone data store or as a cloud data storage. The health data repository (106) may receive the plurality of user and user's health related information from the HMS (102) upon being transmitted by the user device (104) and/or the admin device (108).

The user device (104) may be a mobile device or a personal device or a tablet or a computing device including the functionality for communicating with HMS (102) over the communication network (110). In one embodiment, the user device (104) is configured with a user application (116) that enables the user for example, an employee of an organization to interact with the HMS (102) to request for assessing health risk of the employee. For example, the user application (116) is configured to display at least one set of interactive questionnaires to receive the clinical symptomatic information of the user in response to the set of interactive questionnaires. The user application (116) is further configured to record cough sounds of the user via an acoustic sensor (118) integrated within the user device (104) and transmit the recorded cough sounds to the HMS (102) via the communication network (110). The acoustic sensor (118) may be for example, an audio recorder or a microphone embedded in the user device (104) to record cough sounds of the user. The cough sounds captured by the acoustic sensor (118) may be stored by the HMS (102) in the health data repository (106).

In an alternate embodiment, the user device (104) is also configured to communicate with one or more portable health parameter measuring devices such as the temperature sensor (112-1), the SPO2 sensor (114-1) etc., via one of plurality of modes of communication such as Bluetooth communication, Near-Field Communication (NFC), wired communication etc. The temperature sensor (112-1) may be any device that collects temperature data from a particular source and converts the data into understandable format for the user, wherein the temperature sensor (112-1) may be for example, an electronic thermometer, IR (Infrared) thermometer, Laser thermometer etc. The temperature sensor (112-1) is capable of measuring body temperature of the user and transmitting the measured body temperature to the user device (104). The temperature sensor (112-1) transmits the measured body temperature of the user to the user application (116) for further processing by the HMS (102). In one embodiment, the HMS (102) processes the measured body temperature and determines a temperature risk index for the user by comparing the measured body temperature with a pre-defined threshold value for body temperature.

The SPO2 sensor (114-1) may be any device capable of estimating the oxygen saturation level on the blood of a user. In one embodiment, the SPO2 sensor (114-1) measures the percentage of blood that is loaded with oxygen for the user. Example of SPO2 sensor may be disposable SPO2 sensor, fingertip SPO2 sensor, auricular SPO2 sensor, foot SPO2 sensor, toe SPO2 sensor etc. The user device (104) may be coupled to the user's SPO2 sensor (114-1) available at home to measure oxygen saturation level of blood of the user. The SPO2 sensor (114-1) further transmits the measured oxygen saturation level to the user application (116) via one of plurality of modes of communication such as Bluetooth communication, NFC, wired communication etc. for further processing by the HMS (102). In one embodiment, the HMS (102) determines an oxygen saturation risk index for the user by comparing the measured oxygen saturation level with a pre-defined threshold value for oxygen saturation and stores the oxygen saturation level and the oxygen saturation risk index in the health data repository (106).

The user application (116) is also configured to receive information related to one or more health parameters from the respective portable health parameter measuring devices via the user device (104) and transmit the one or more health parameter measurement information to the HMS (102) via the communication network (110). The HMS (102) determines the final risk index to assess health risk of the user based on the combined analysis of all the measured health parameters. The user application (116) can also be configured to enable the user to manually enter measurements of one or more health parameters such as body temperature, oxygen saturation level etc., so as to facilitate the HMS (102) to determine risk associated with user health.

The user application (116) can be configured as a user end application that manages a plurality of health assessment requests received from the user i.e., employees of the organization. The user application (116) can also be configured to store plurality of user data (120) such as user profile, preferences etc. In one embodiment, the user application (116) is configured to facilitate the user to register user profile and preferences, respond to the interactive questionnaires, and receive plurality of analytical health information. User preferences can be dashboard view of risk information, preferred selection of one or more analytical information etc. In an example, health risk assessment can be initiated by the user, wherein the user application (116) provides a set of interactive questionnaires to the user and the user can respond to such questionnaires for enabling the HMS (102) to record clinical symptoms of the user and determine symptom risk index. The user application (116) also provides automated notification to the user for measurement of one or more health parameters such as cough sound, body temperature, oxygen saturation etc., based on the clinical symptoms. The user application (116) further enables the user to receive a final risk index indicative of estimated health risk information of the user based on plurality of health parameters.

The admin device (108) may be a mobile device or a tablet or a computing device including the functionality for communicating over the communication network (110), wherein the admin device (108) is operable at workplace coupled along with the temperature sensor (112-2) and the SPO2 sensor (114-2). The temperature sensor (112-2) may be any device that collects temperature data from a particular source and converts the data into understandable format for the user, wherein the temperature sensor (112-2) may be for example, an electronic thermometer, IR (Infrared) thermometer, Laser thermometer etc. In an example, the temperature sensor (112-2) may be installed at different entry point of an office premises of an organization so as to monitor body temperature of plurality of employees of the organization. The temperature sensor (112-2) is capable of measuring body temperature of the user and transmitting the measured body temperature to the admin device (108).

The SPO2 sensor (114-2) may be any device capable of estimating the oxygen saturation level on the blood of the employees. Example of SPO2 sensor (114-2) may be a disposable SPO2 sensor, fingertip SPO2 sensor, auricular SPO2 sensor, foot SPO2 sensor, toe SPO2 sensor etc. The SPO2 sensor (114-2) may be installed at different entry points of an office building of an organization so as to measure oxygen saturation level of blood of the plurality of employees of the organization. The SPO2 sensor (114-2) further transmits the measured oxygen saturation level to the admin application (122) via one of plurality of modes of communication such as Bluetooth communication, NFC, wired communication etc. for further processing by the HMS (102). In one embodiment, the SPO2 sensor (114) measures the percentage of blood that is loaded with oxygen for the employees. In one embodiment, the HMS (102) determines the oxygen saturation risk index for the employees by comparing the measured oxygen saturation level with a pre-defined threshold value for oxygen saturation and stores the oxygen saturation level and the oxygen saturation risk index in the health data repository (106).

The admin device (108) is configured with an admin application (122) that receives body temperature reading and oxygen saturation level of an employee as measured by the temperature sensor (112-2) and the SPO2 sensor (114-2) respectively. In one embodiment, the admin application (122) is configured initiates the measurement of the body temperature reading and oxygen saturation level of the employee only upon receiving a unique code from the user or employee via the user device (104). The user device (104) transmits the unique code generated by the HMS (102) in response to receiving health related information like symptoms data and cough sounds from the user device (104) when the user initiated a request for monitoring health risk. The unique code may be for example, a QR code, barcode etc., and may be a unique signature indicating the combination of user identification information, and user's corresponding symptoms data and cough sounds. The admin application (122) is configured to identify the received unique code provided by the user and initiates the measurement of health parameters from the one or more measuring devices upon successful matching with the unique code of the same user stored in the HMS (102).

In operation, the user may first initiate the health assessment for one or more health parameters that can be measured via the user device (104) at home, and upon completion of such assessment, the user receives the unique code for the assessment of one or more other health parameters at the workplace. The user may use the same unique code to record assessment of plurality of health parameters like temperature and oxygen saturation at the workplace so as to enable the HMS (102) to perform the combined analysis of all the health parameters every time the user wishes to monitor health risk.

In one embodiment, the generated unique code can further be used in a health diagnostic centre, wherein the measuring devices are pre-configured to communicate with HMS (102). In an example, the user receives an QR (Quick Response) code in the user application (116) upon initiating the health risk assessment using the user device (104). The HMS (102) determines the user as safe to work based on the symptom analysis and cough sound analysis. The admin device (108) scans the QR code via a scanner while entering the workplace. Upon scanning the QR code, the body temperature and oxygen saturation level of the user are measured using the temperature sensor (112-2) and SPO2 sensor (114-2) respectively installed at the workplace. The admin device (108) receives the body temperature reading and oxygen saturation level from the temperature sensor (112-2) and the SPO2 sensor (114-2) and transmits the same to the HMS (102).

The admin application (122) can be configured as an admin end application that manages the health parameter measurements for one or more users. In one embodiment, the admin application (122) is configured to facilitate the admin to retrieve personal details of the user for identification purpose. The admin application (122) further enables the admin to provide manual input to the health parameter measurements in case of any communication disruption between the admin device (108) and the temperature sensor (112-2) or the SPO2 sensor (114-2). In another embodiment, the admin application (122) allows the administrator of the organization to perceive a plurality of health data/information related to health risk of a group of employees, wherein the plurality of analytical information includes categorization of safe and unsafe users, weekly risk trend analysis, longitudinal view of each symptom for each employee etc. The admin application (122) facilitates the administrator with closely monitored information on the symptoms of the employees on daily or even hourly basis in the respective user interface. In one embodiment, the admin device (108) is further capable of being configured with one or more other health parameter measuring device such glucometer, heart rate monitor etc. Therefore, the HMS (102) can determine health risk by incorporating one or more other health parameters such as blood sugar, heart rate etc., wherein incorporation of such other health parameters results into more accurate health risk assessment. Upon determining the symptom risk index and cough risk index, the HMS (102) determines the user as safe-to-work or not safe-to-work and enables the user to proceed with the measurement of other health parameters including but not limited to body temperature, oxygen saturation level, respiratory rate, blood components etc., based on safe-to-work or not safe-to-work condition of the user. The HMS (102) further determines the risk associated with each of the other parameters upon comparison of measured value with the pre-defined threshold value for the respective parameter and generates the final risk index based on the risk indices of all the health parameters, thereby assessing the health risk more accurately. Therefore, the HMS (102) can be configured according to the type of health risk monitoring required for different type of adverse conditions that are detrimental to public health.

The HMS (102) may be configured as a cloud-based implementation server or as a standalone server. In one embodiment, the HMS (102) comprises a processor (130) and a memory (132) coupled to the processor (130) that stores processor-executable instructions. The processor (130) and the memory (132) are communicatively coupled to the user device (104), the health data repository (106) and the admin device (108). The HMS (102) further comprises one or more modules configured to monitor health risk of the user in real time. In one embodiment, the one or more modules include a data acquisition module (134), a cough analysis module (136), and a risk assessment module (138). The HMS (102) is configured to facilitate the user to provide input regarding clinical symptoms, cough sounds, body temperature and oxygen saturation level and estimate a final risk index of the user's health based on combined analysis of the aforementioned inputs. The HMS (102) further facilitates the administrator of the organization by providing plurality of notifications and analytical information regarding the health condition of a group of users so that the administrator can enforce suitable restriction to ensure safety to other people within the organization. Therefore, the HMS (102) provides a health monitoring system performing combined analysis of critical health parameters and predicting health insights.

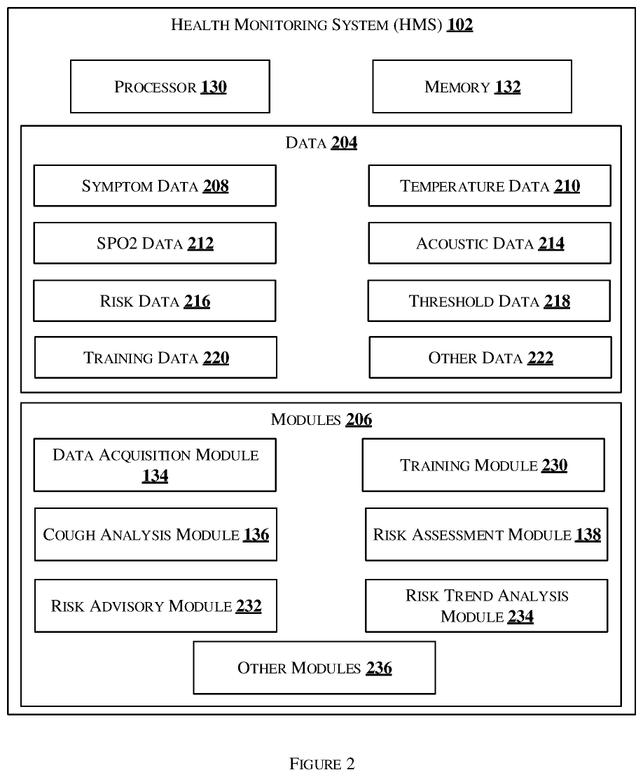

As illustrated in FIG. 2, the HMS (102) may be a typical health monitoring system. In one embodiment, the HMS (102) includes data (204) and modules (206). In one embodiment, the data (204) can be stored within the memory (132). In one example, the data (204) may include symptom data (208), temperature data (210), SPO2 data (212), acoustic data (214), risk data (216), threshold data (218), training data (220), and other data (222). In one embodiment, the data (204) can be stored in the memory (132) in the form of various data structures. Additionally, the aforementioned data can be organized using data models, such as relational or hierarchical data models. The other data (222) may be also referred to as reference repository for storing recommended implementation approaches as reference data. The other data (222) may also store data, including temporary data, temporary files, intermediate process data, notification data, learning data generated by the modules (206) for performing the various functions of the HMS (102).

The modules (206) may include, for example, the data acquisition module (134), a training module (230), the cough analysis module (136), the risk assessment module (138), a risk advisory module (232) and a risk trend analysis module (234). The modules (206) may also comprise other modules (236) to perform various miscellaneous functionalities of the HMS (102). It will be appreciated that such aforementioned modules may be represented as a single module or a combination of different modules. The modules (206) may be implemented in the form of software performed by the processor, hardware and/or firmware.

In one embodiment, the data acquisition module (134) receives user response related to the interactive questionnaire, wherein the questionnaire is a predefined set of questions based upon plurality of clinical symptoms. The predefined set of questions can include user's travel history, pre-existing ailments, cough related symptoms, one or more common symptoms associated with highly infectious pandemic diseases etc. In an example, the plurality of basic symptoms include but not limited to fatigue or tiredness, loss of smell, loss of taste, running nose, sneezing, sore throat, headache, nasal congestion, nausea vomiting etc. The cough related symptoms include cough frequency, cough at night, sputum in cough, wheezing, chest pain, difficulty in breathing etc. The pre-existing ailments include but not limited to diabetes, heart disease, lung disease, stroke, kidney problem, weak immune system, pregnancy, major operations like organ transplant, obesity, smoking habit etc. The data acquisition module (134) stores the user response to the interactive questionnaire as the symptom data (208) in the health data repository (106). The data acquisition module (134) further receives the plurality of cough sounds recorded by the acoustic sensor (118) in a regular interval and stores the received plurality of cough sounds along with timestamp as acoustic data (214) in the health data repository (106). The data acquisition module (134) receives the body temperature of the user as input captured by the temperature sensor (112) and stores the captured body temperature along with timestamp as temperature data (210) in the health data repository (106). The data acquisition module (134) further receives oxygen saturation information measured by the SPO2 sensor (114) and stores the received oxygen saturation information along with timestamp as SPO2 data (212) in the health data repository (106).

In one embodiment, the data acquisition module (134) stores a plurality of training datasets as training data (220) for training the machine learning model, wherein the plurality of training datasets is received from one or more external sources. The plurality of training datasets includes historical information related to cough sounds, cough pattern, pattern severity, underlying respiratory condition, cough severity score, dry cough count, wet cough count etc. of a group of healthy people and a group of people suffering from one or more respiratory diseases. The data acquisition module (134) stores one or more pre-defined threshold values as threshold data (218) in the health data repository (106), wherein the one or more pre-defined threshold values may include medically defined standard ranges of body temperature, oxygen saturation level of a human body. In another embodiment, the data acquisition module (134) is configured to accumulate the interactive questionnaire response, cough sounds and other cough information, body temperature, oxygen saturation information for one or more users over a period of time and store the accumulated information in the health data repository (106).

The training module (230) retrieves the training data (220) from the health data repository (106) and performs a process of training the machine learning model by feeding the training data (220) to the machine learning model. The training module (230) retrieves the historical information related to cough severity score, dry cough count, wet cough count of the group of healthy people and a group of unhealthy people suffering from respiratory disease from the training data (220). The training module (230) further defines a first set of pre-defined threshold values and a second set of pre-defined threshold values for each of cough severity score, dry cough count, and wet cough based on the analysis of the retrieved historical information of the group of healthy people and the group of unhealthy people respectively. The first set of pre-defined threshold values define the acceptable upper limits of the cough severity score, dry cough count and wet cough count for healthy users. The training module (230) determines risk of the user if any of measured cough severity score, dry cough count, and wet cough count is determined to exceed the first set of pre-defined threshold values defined for the healthy person. The second set of pre-defined threshold values define the acceptable lower limits of the cough severity score, dry cough count and wet cough count for any unhealthy users suffering from respiratory disease. The training module (230) determines risk of the user if any of measured cough severity score, dry cough count, and wet cough count is determined to exceed the second set of pre-defined threshold values defined for the user suffering from respiratory disease.

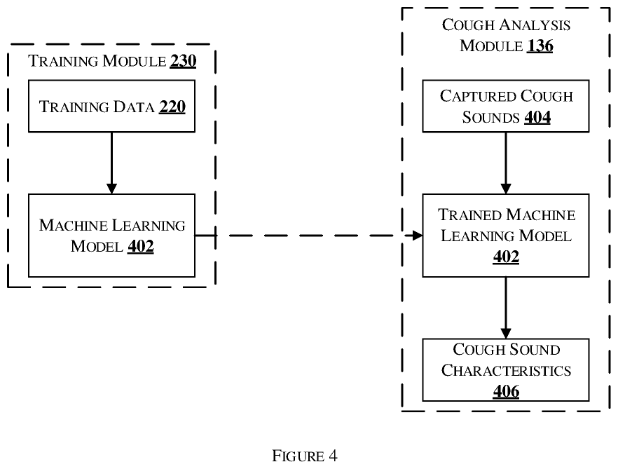

The training module (230) uses the training data (220) to train the machine learning model (402) as illustrated in FIG. 4. The training module (230) processes the training data (220) in one or more phases to prepare a training dataset and a testing dataset, wherein the one or more phases include but not limited to analysis of data, handling missing data, cleansing of data, deciding key factors related to data etc. The training module (230) further splits the training set into a plurality of mini batches so that the process of training can be conducted in a plurality of iterations. Once the machine learning model (402) receives each of the mini-batches as input, the machine learning model (402) initiates the learning from the information contained in each of the mini-batches, wherein the machine learning model (302) can use one of plurality of learning mechanism such as supervised learning, unsupervised learning etc. The training of the machine learning model (402) is completed upon processing all the mini batches. Further, the training module (230) performs testing of the trained machine learning model (402) using the testing set to ensure desired performance of the machine learning model (402). The trained machine learning model further receives captured cough sounds (404) of the user and determines one or more cough sound characteristics (406) based on the imparted training. In one embodiment, upon completion of the initial training process, the machine learning model (402) continuously learns from the processing of one or more received cough sounds in real time.

The cough analysis module (136) retrieves the plurality of cough sounds of the user from the acoustic data (214) and derives one or more cough sound characteristics from the retrieved cough sounds, wherein the one or more cough sound characteristics include but not limited to underlying respiratory condition, cough pattern, pattern severity, dry cough severity, wet cough severity etc. The cough analysis module (136) analyses each of the retrieved plurality of cough sounds by using the trained machine learning model (402). The trained machine learning model (402) receives the plurality of cough sounds as input and determines one or more cough characteristics including underlying respiratory condition, cough pattern, pattern severity based on learning from the training data (220). The cough analysis module (136) computes one or more cough sequences in each of the retrieved plurality of cough sounds, and number of coughs in each of the computed cough sequences. The cough analysis module (136) determines total count of coughs in each of the retrieved plurality of cough sounds based on computed number of cough sequences and number of coughs in each cough sequence. Upon determining the total count of coughs, the cough analysis module (136) identifies type of cough as one of dry cough and wet cough for each cough sound in the retrieved plurality of cough sounds and computes at least a dry cough count and a wet cough count for each of the plurality of cough sounds based on total count of coughs and identified type of each cough of plurality of cough sounds. The cough analysis module (136) further computes a dry cough severity as one of low and high and a wet cough severity as one of low and high for each of the plurality of cough sounds based on the comparison of the dry cough count and the wet cough count with respective one of the first set and the second set of pre-defined threshold values as determined by the training module (230). The cough analysis module (136) stores the one or more cough characteristics including dry cough count, dry cough severity, wet cough count, and wet cough severity as the acoustic data (214) in the health data repository (106).

In an example, the first set of pre-defined threshold values for dry cough count and wet cough count are 10 and 8 respectively, and the computed dry cough count and wet cough count of a healthy user are 14 and 7 respectively. Therefore, the dry cough severity is determined as high as the dry cough count exceeds the first set of pre-defined threshold value and the wet cough severity is determined as low as the wet cough count is within the first set of pre-defined threshold value.

In another example, the second set of pre-defined threshold values for dry cough count and wet cough count are 15 and 12 respectively, and the computed dry cough count and wet cough count of an unhealthy user suffering from respiratory disease are 17 and 8 respectively. Therefore, the dry cough severity is determined as high as the dry cough count exceeds the second set of pre-defined threshold value and the wet cough severity is determined as low as the wet cough count is within the second set of pre-defined threshold value.

In one embodiment, the cough analysis module (136) is further configured to determine a set of individual threshold values for the cough severity score, dry cough count, and wet cough count. The cough analysis module (136) retrieves the periodically stored cough severity scores, dry cough counts, and wet cough counts of the user from the acoustic data (214) and determines a user specific or individual threshold for each of cough severity score, dry cough count, and wet cough based on the analysis of the retrieved periodic information. The individual threshold values define the acceptable upper limits of the cough severity score, dry cough count and wet cough count of that particular user, and the individual threshold value differ from one person to another person.

In another embodiment, the cough analysis module (136) is configured to dynamically update the first set and the second set of pre-defined threshold values and the individual threshold values upon receiving further cough information with respect to the one or more new users by using the machine learning model. The machine learning model recalculates the individual threshold values of cough severity score, dry cough count, and wet cough count when the data acquisition module (134) receives more cough sounds from the user. Further, the machine learning model recalculates the first set and the second set of pre-defined threshold values of cough severity score, dry cough count, and wet cough count in a scheduled interval upon receiving and processing of cough sounds of new users of HMS (102) by the data acquisition module (134).

The risk assessment module (138) retrieves one or more user responses to the interactive questionnaires from the symptom data (208) and determines plurality of clinical symptoms based on the responses of the user. The risk assessment module (138) assigns a pre-defined weightage score to each of the plurality of clinical symptoms data and computes a total weightage symptom score based on summation of pre-defined weightage score to each of the clinical symptoms data. Upon determining the total weightage symptom score, the risk assessment module (138) determines a symptom risk index as one of ‘1’ and ‘0’ based on comparison of total weightage symptom score with a pre-defined threshold weightage symptom score and stores the total weightage symptom score and the symptom risk index as the risk data (216). The pre-defined threshold weightage score is determined based on the medical guidelines related to one or more infectious diseases as published by one or more health regulatory bodies such as WHO (World Health Organization), government health department etc. The medical guidelines provide severity of one or more symptoms, threshold for such symptoms etc., in a regular interval based on the criticality of such infectious diseases and other related conditions such as impact to human being, sustainability of infection etc. In one example, a total weightage symptom score exceeding the pre-defined threshold weightage score is considered as risky health condition of the user.

As an example, conditions like travel out of country, contact with person travelled to infected areas are assigned with weightage 1, wherein clinical symptoms like shortness of breath, loss of smell, loss of taste are assigned with weightage 0.5; headache, sore throat are assigned with 0.15; nausea, nasal congestion are assigned with 0.1 etc. If a person has travelled outside and has shortness of breath, then the total weightage of the person is 1+0.5 i.e. 1.5 and the total weightage symptoms score is computed by multiplying the total weightage by 10 (i.e. 1.5×10=15) and rounding off the multiplication value (i.e. 15) of total weightage to 10. Therefore, the total symptom severity score is computed as 10 and the symptom risk index is determined as ‘1’.

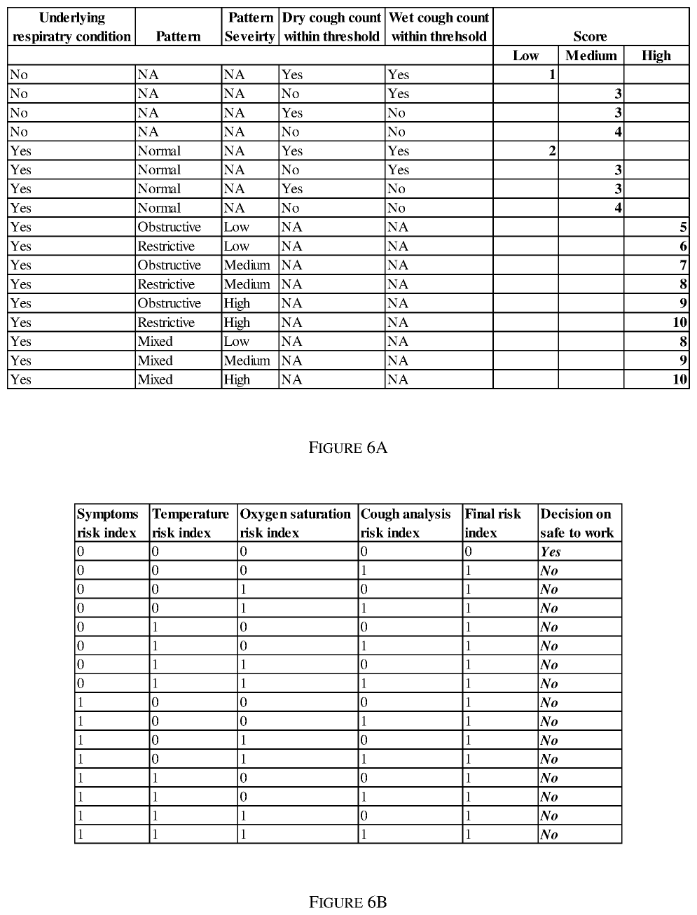

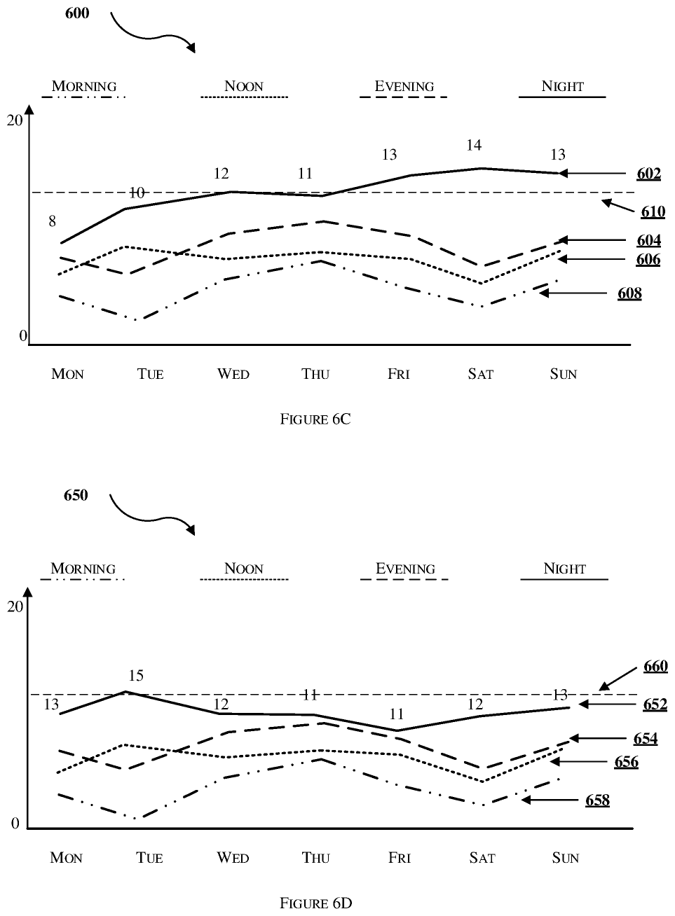

The risk assessment module (138) further retrieves the cough sound characteristics from the acoustic data (214) and performs a combined analysis of the retrieved cough sound characteristics to determine a cough severity score. The risk assessment module (138) can determine the cough severity score for a plurality of possible combination of values of underlying respiratory condition, cough pattern, pattern severity, dry cough severity, and wet cough severity based on a predefined mapping of the values of cough sound characteristics and the cough severity score. In the predefined mapping as illustrated in the cough severity scoring table of FIG. 6A, the value of underlying respiratory condition can be one of ‘Yes’ or ‘No’, the value of cough pattern can be one of normal obstructive, restrictive, mixed etc., the value of pattern severity can be one or low, medium and high. The cough severity score is defined in a range from 1 to 10 based on the plurality of combination of values of underlying respiratory condition, cough pattern, pattern severity, dry cough severity, and wet cough severity. The risk assessment module (138) retrieves one of the first and the second pre-defined threshold values of the cough severity score based on the underlying respiratory condition. The risk assessment module (138) further determines a cough risk index as one of ‘0’ and ‘1’ based on the analysis of the cough severity score with respect to the one of the first and the second pre-defined threshold values, wherein the cough risk index is determined as ‘1’ when the cough severity score exceeds 2. The risk assessment module (138) further stores the cough risk index and the cough severity score as the risk data (216).

As an example, the user's cough sound characteristics are as the following:
1. Underlying respiratory problem—Yes
2. Cough Pattern—Normal
3. Pattern Severity—Low
4. Dry cough count within threshold—Yes (i.e. dry cough severity is low)
5. Wet cough count within threshold—Yes (i.e. wet cough severity is low)

Therefore, the cough severity score is determined as 2 (i.e. cough severity is low) based on the pre-defined mapping as illustrated in FIG. 6A and the cough risk index is determined as low based on the aforementioned parameters. In another example, the user's cough sound characteristics are as the following:
1. Underlying respiratory problem—Yes
2. Cough Pattern—Normal
3. Pattern Severity—Low
4. Dry cough count within threshold—No (i.e. dry cough severity is high)
5. Wet cough count within threshold—No (i.e. wet cough severity is high)

The cough severity score is determined as 4 (i.e. cough severity is medium) based on the pre-defined mapping as illustrated in FIG. 6A and the cough risk index is determined as ‘1’ based on the aforementioned parameters.

The risk assessment module (138) retrieves the captured temperature information from the temperature data (210) and compares the captured temperature information with the pre-defined threshold for body temperature. The risk assessment module (138) determines a temperature risk index as one of ‘0’ and ‘1’ based upon the comparison of captured temperature information and the pre-defined threshold for body temperature. The risk assessment module (138) defines a temperature severity as one of low, medium, and high based on the captured temperature information and stores the temperature risk index and the temperature severity as the risk data (216). In one example, the captured temperature reading of the user is 103.0° F. and the pre-defined threshold for body temperature is 100.4° F. Therefore, the temperature risk index of the user is 1 as the captured temperature exceeds the pre-defined threshold and the temperature severity is defined as high.

Upon determining the temperature risk index and temperature severity, the risk assessment module (138) further retrieves the oxygen saturation information from SPO2 data (212) and analyses the oxygen saturation information with respect to the pre-defined threshold of oxygen saturation level of human body. The risk assessment module (138) determines an oxygen saturation risk index as one of ‘0’ and ‘1’ based upon the analysis of retrieved oxygen saturation information and defines an oxygen saturation severity as one of low, medium, and high based on the different ranges of oxygen saturation level. The risk assessment module (138) further stores the oxygen saturation risk index and the oxygen saturation severity as risk data (216) of health data repository (106).

In another example, the oxygen saturation level of 92% and above is considered as normal for healthy people, wherein the oxygen saturation level between 85% and 92% indicates hypoxia and the oxygen saturation level below 85% indicates severe hypoxia. If the measured oxygen saturation level of the user is 90%, the user oxygen saturation risk index is determined as 1 and the oxygen saturation severity is defined as medium.

In one embodiment, the risk assessment module (138) determines a final risk index of the user's health based upon the combined analysis of determined symptom risk index, cough risk index, temperature risk index, and oxygen saturation risk index. The final risk index facilitates the HMS (102) to automatically assess the health risk of the user as one of safe-to-work and not safe-to-work based on the final risk index. The risk assessment module (138) determines the final risk index as ‘1’ when at least one of the symptom risk index, cough risk index, temperature risk index, and oxygen saturation risk index is determined as ‘1’ and further determines that the user is not safe-to-work. As illustrated in the final risk index scoring table of FIG. 6B, the final risk index is determined based on the combined analysis of the symptom risk index, cough risk index, temperature risk index, and oxygen saturation risk index, wherein all the risk indices are represented with value ‘0’ or ‘1’. The value 0 represents low risk and value 1 represents high risk. The FIG. 6B further explains a mapping of different possible values of the final risk index with the different combination of risk index values of the aforementioned health parameters. Based on the mapping, the final risk index is determined as ‘1’ when at least one of the symptom risk index, cough risk index, temperature risk index, and oxygen saturation risk index is ‘1’. The final risk score of ‘0’ or ‘1’ further facilitates in decision on safe-to-work decision as ‘Yes’ and ‘No’ respectively.

The risk advisory module (232) generates an alert or notification upon determination of health risk of the user as not safe-to-work if the final risk score is determined to be ‘1’. The risk advisory module (232) generates alert that contains details of the associated risk in one or more formats suitable for one or more transmission modes such as SMS (Short Message Services), email, app notification, automated phone calls etc. The risk advisory module (232) transmits the generated alert or notification along with the symptom risk index, cough risk index, temperature risk index, oxygen saturation risk index and final risk index to the user and/or one or more authorized persons related to the user. The risk advisory module (232) further generates one or more actions items recommended for respective health risk such as symptom risk, cough risk, temperature risk, oxygen saturation risk based on the predefined actions items stored in the health data repository (106). The risk advisory module (232) is configured to predefine one or more action items that must be acted upon based on different possible categories of health risk and store in the health data repository (106). The risk advisory module (232) further notifies the one or more recommended action items to the user and/or the one or more authorized person related to the user by using a suitable transmission mode. The risk advisory module (232) receives updates from the user or one or more authorized person via app-based progress tracker, SMS (Short Message Services), automated confirmation call etc., wherein such updates are very crucial for avoiding the health risk of the user. The risk advisory module (232) determines the progress on the recommended action items based on the received updates on such recommended action items. Upon determining incomplete status of the recommended action items, the risk advisory module (232) further transmits the pending recommended action items to the user or one or more authorized person related to the user. The risk advisory module (232) stops transmitting the recommended action items upon determination of completion of the previously issued recommended action items. In one embodiment, the risk advisory module (232) also generates notification for the administrator upon determination of health risk of the user as safe-to-work.

In one embodiment, the risk trend analysis module (234) retrieves the stored temperature reading and oxygen saturation level from the temperature data (210) and SPO2 data (212) respectively and determines trends of temperature reading and oxygen saturation level of the user over the period of time with respect to the pre-defined threshold values. The risk trend analysis module (234) predicts a surge of temperature reading or decline in oxygen saturation based on the determined trends and generates alerts to the user or one or more authorized person related to the used based on the prediction.

The risk trend analysis module (234) further retrieves the stored cough severity score from the acoustic data (214) and determines trends of cough severity score of the user over the period of time with respect to the first and the second set of pre-defined threshold values. The risk trend analysis module (234) further predicts a surge in cough severity score based on the determined trends and generates alerts to the user or one or more authorized person related to the used based on the prediction.

The automatic prediction-based alert generation based on the trend of one or more health parameters enables the user and one or more healthcare professionals to determine preventive measures without having further deterioration of the user's health.

In one embodiment, the risk trend analysis module (234) retrieves plurality of information, related to one or more health parameters of the user or a group of users, such as clinical symptoms, cough sounds, body temperature, oxygen saturation information etc. from the health data repository (106) for a period of time or a specific point of time. Upon retrieving the plurality of health information of the user or group of users, the risk trend analysis module (234) generates a plurality of analytical information for the user or the administrator of the organization, wherein such analytical information includes but not limited to longitudinal view for each symptom, weekly or monthly risk trend, variation in body temperature, average respiratory condition of group of users etc. The risk trend analysis module (234) further enables the administrator of any organization to perceive a summarized view of plurality of health information of plurality of users.

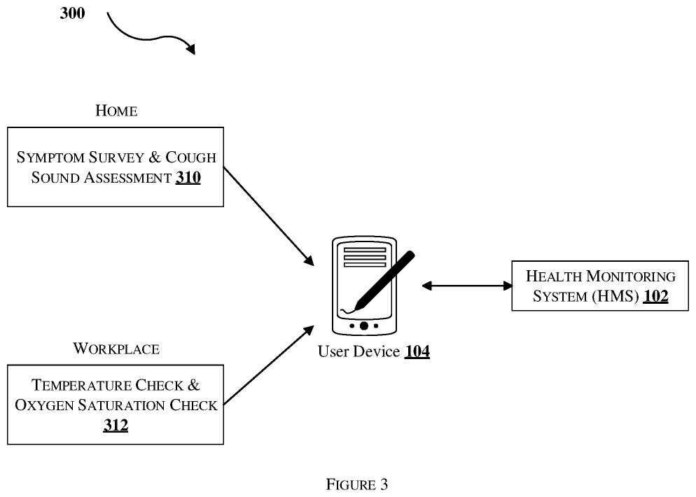

FIG. 3 illustrates an exemplary representation for stepwise health risk assessment in accordance with some embodiments of the present disclosure.

In an alternate embodiment, the HMS (102) is configured to perform stepwise health risk assessment. In the stepwise assessment, the user performs symptom survey and cough sound assessment (310) by staying at home using the user device (104) as a first step, and performs temperature check and oxygen saturation check (312) by using the temperature sensor (112-2) and SPO2 sensor (114-2) installed at workplace as a second step of health assessment. The information related to clinical symptoms and cough sounds are transmitted to the HMS (102) via the user device (104). The risk assessment module (138) determines a symptom risk index by analysis of the user response to the interactive questionnaire. The cough analysis module (136) determines one or more cough sound characteristics based on the analysis of received cough sound. Upon determination of one or more cough sound characteristics, the risk assessment module (138) determines a cough severity score and a cough risk index. The risk assessment module (138) determines a final risk index as one of ‘0’ and ‘1’ based on the symptom risk index and the cough risk index. The risk assessment module (138) further determines the user as safe-to-work or not safe-to-work based on the determined final risk index. Upon determination of the user as safe-to-work, the user's body temperature and oxygen saturation level are further measured as additional health risk parameters at workplace via the admin device (108). The admin device (108) enables the measurement of the body temperature reading and the oxygen saturation level of the user via the temperature sensor (112-2) and SPO2 sensor (114-2) installed at the workplace and transmit the measurements to the HMS (102) via the admin application (122). The risk assessment module (138) determines a temperature risk index and an oxygen saturation risk index based on the analysis of measured body temperature and oxygen saturation level. The risk assessment module (138) further determines another final risk index as one of ‘0’ and ‘1’ based on combined analysis of health parameters examined at both home and workplace, thus resulting into more accurate health risk assessment.

The stepwise assessment enables the user to determine health risk based upon essential check like the symptom survey and the cough sound assessment without assessing the temperature risk and the oxygen saturation level risk. Therefore, such assessment enables quick and effective screening at less time and eliminates the requirement of further measurements like temperature check and oxygen saturation check in case of a risk is already determined by the HMS (102) based on the symptom survey and cough sound assessment (310). As an example, the user starts health risk assessment by responding to the plurality of questionnaire related to clinical symptoms of the user like his international travel and providing a plurality of cough sounds. Upon receiving the clinical symptoms data and the plurality of cough sounds, the HMS (102) determines the symptom risk index as ‘1’ due to his recent travel history. Therefore, the HMS (102) determines that the user is not safe-to-work and the temperature check and oxygen saturation check at workplace can be skipped as the user may suffer from infectious disease which can infect others at workplace or the user' health may further deteriorate due to external exposure.

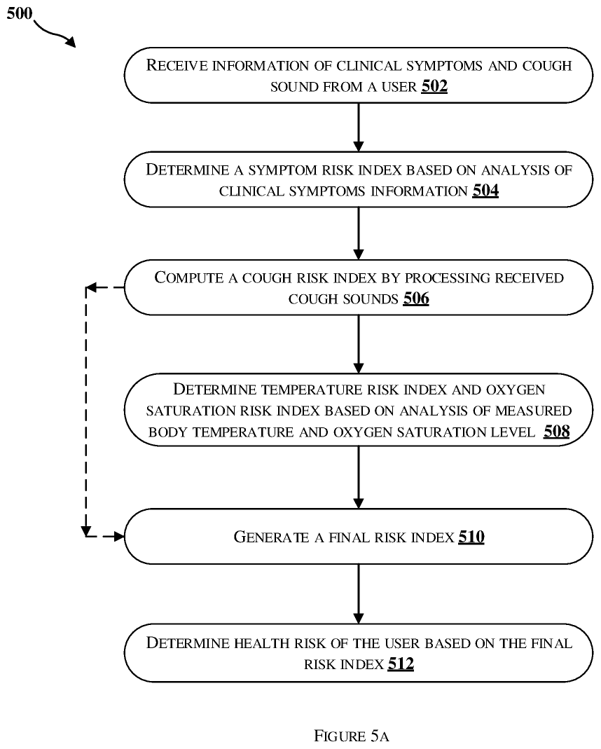

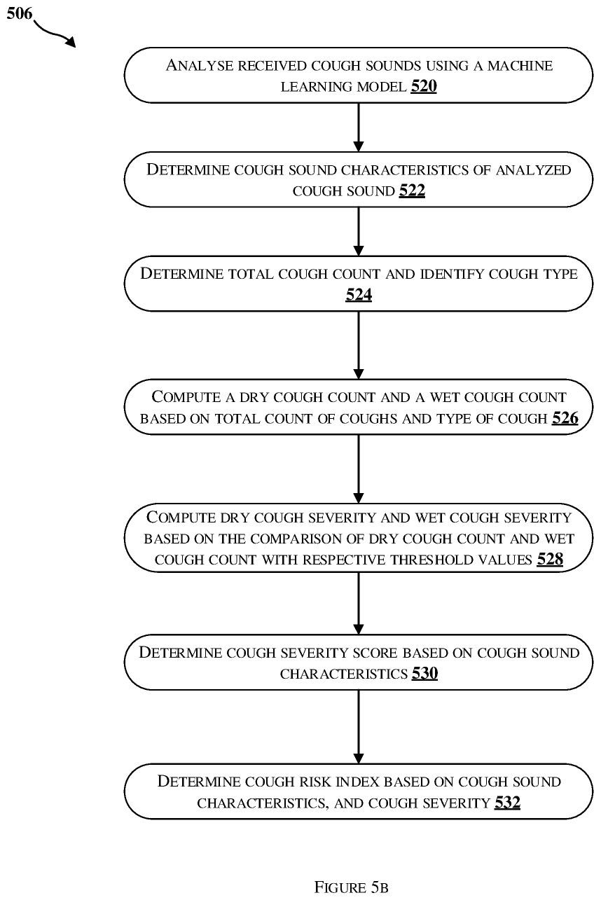

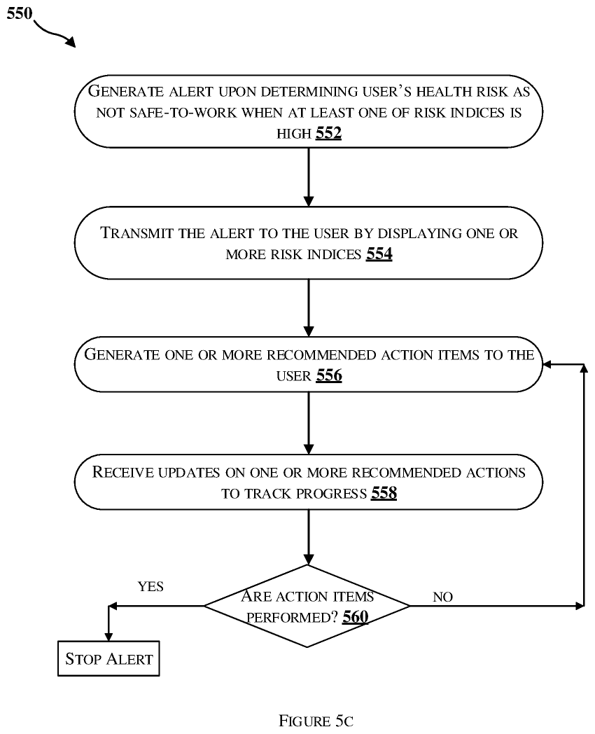

FIG. 5A illustrates a flowchart showing a method for monitoring health risk in accordance with some embodiments of the present disclosure.

As illustrated in FIG. 5A, the method (500) comprises one or more blocks implemented by the processor (130) to monitor health risk by using HMS (102). The method (500) may be described in the general context of computer executable instructions. Generally, computer executable instructions can include routines, programs, objects, components, data structures, procedures, modules, and functions, which perform specific functions or implement specific abstract data types.

The order in which the method (500) is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

At block (502), clinical symptoms data and cough sounds are received from the user. In one embodiment, the data acquisition module (134) receives user response related to the interactive questionnaire, wherein the questionnaire is a predefined set of questions based upon plurality of clinical symptoms and stores the received user response as symptom data (208) of health data repository (106). The predefined set of questions can include user's travel history, pre-existing ailments, cough relates symptoms, one or more basic symptoms etc. The data acquisition module (134) further receives the plurality of cough sounds recorded by the acoustic sensor (118) in a regular interval and stores the received plurality of cough sounds as acoustic data (214) in the health data repository (106).

At block (504), the symptom risk index is determined based on the analysis of clinical symptoms data. In one embodiment, the risk assessment module (138) retrieves one or more user responses to the interactive questionnaires from the symptom data (208) and determines plurality of clinical symptoms based on the responses of the user. The risk assessment module (138) assigns a pre-defined weightage score to each of the plurality of clinical symptoms data and computes a total weightage symptom score based on summation of pre-defined weightage score to each of the clinical symptoms data. Upon determining the total weightage symptom score, the risk assessment module (138) determines a symptom risk index as ‘0’ and ‘1’ based on comparison of total weightage symptom score with the pre-defined threshold of weightage symptom score and stores the total weightage symptom score and the symptom risk index as the risk data (216).

At block (506), the cough risk index is computed by processing of cough sound. In one embodiment, the cough analysis module (136) retrieves the plurality cough sounds of the user from the acoustic data (214) and derives one or more cough sound characteristics from the retrieved cough sounds, wherein the one or more cough sound characteristics include but not limited to underlying respiratory condition, cough pattern, pattern severity, dry cough severity, wet cough severity etc. The cough analysis module (136) stores the determined one or more cough characteristics including the dry cough count and the wet cough count as acoustic data (214) in the health data repository (106). The risk assessment module (138) further retrieves the cough sound characteristics from the acoustic data (214) and performs a combined analysis of the retrieved cough sound characteristics to determine the cough severity score. The risk assessment module (138) determines the cough risk index as one of ‘0’ and ‘1’ based on the analysis of the cough severity score and stores the cough risk index and the cough severity score as the risk data (216).

At block (508), the temperature risk index and the oxygen saturation risk index are determined based on analysis of measured body temperature and oxygen saturation level of the user. In one embodiment, the data acquisition module (134) receives the user's body temperature as measured by the temperature sensor (112-2) and oxygen saturation information as measured by the SPO2 sensor (114-2). The data acquisition module (134) further stores the temperature information and oxygen saturation information as temperature data (210) and SPO2 data (212) respectively. The risk assessment module (138) retrieves the captured temperature information from the temperature data (210) and compares the captured temperature information with the pre-defined threshold for body temperature. The risk assessment module (138) determines a temperature risk index as one of ‘0’ and ‘1’ based upon the comparison of captured temperature information and the pre-defined threshold for body temperature and stores the temperature risk index as risk data (216). Upon determining the temperature risk index and temperature severity, the risk assessment module (138) further retrieves the oxygen saturation information from SPO2 data (212) and analyses the oxygen saturation information with respect to the pre-defined threshold of oxygen saturation level of human body. The risk assessment module (138) determines an oxygen saturation risk index as one of ‘0’ and ‘1’ based upon the analysis of retrieved oxygen saturation information and stores the oxygen saturation risk index and the oxygen saturation severity as risk data (216) of health data repository (106). The pre-defined threshold values for temperature reading and oxygen saturation level are determined based on medically defined standard ranges.

At block (510), the final risk index is generated. In one embodiment, the risk assessment module (138) determines the final risk index of the user's health as one of ‘0’ and ‘1’ based upon the combined analysis of determined symptom risk index, cough risk index, temperature risk index, and oxygen saturation risk index as illustrated in the mapping of FIG. 6B. The final risk index is determined as ‘1’ when at least any one of symptom risk index, cough risk index, temperature risk index, and oxygen saturation risk index is computed as ‘1’. Therefore, the final risk index indicates a minimum possible risk associated with the user's health.

In one embodiment, the HMS (102) can determine the final risk index based on the determined symptom risk index and cough risk index only as explained in the stepwise assessment in FIG. 3. Therefore, the assessment of body temperature and oxygen saturation level are skipped (as illustrated by connecting step 506 and step 510 via dotted line in FIG. 5A) if the user is determined as not safe-to-work based on the analysis of symptom risk index and cough risk index.

In one embodiment, the HMS (102) can determine the final risk index based on clinical symptoms, cough sound, body temperature, oxygen saturation at home only, wherein the temperature sensor (112-1) and the SPO2 sensor (114-1) are connected to the user device (104) via Bluetooth. The user application (116) of the user device (104) receives the body temperature reading and the oxygen saturation level of the user as transmitted by the temperature sensor (112-1) and the SPO2 sensor (114-1). In analogous approach, one or more other health parameters can be integrated to health risk assessment, wherein the measurement devices of one or more other health parameters are capable of communicating to the user device (104) via Bluetooth.

At block (512), the health risk of the user is determined as one of safe-to-work and not safe-to-work based on the final risk index. In one embodiment, the risk assessment module (138) assesses the health risk of the user as safe-to-work when the final risk index is ‘0’ i.e., all of the symptom risk index, cough risk index, temperature risk index, and oxygen saturation risk index are computed as ‘0’. The risk assessment module (138) assesses the health risk of the user as not safe-to-work when the final risk index is ‘1’ i.e. at least any one of symptom risk index, cough risk index, temperature risk index, and oxygen saturation risk index is computed as ‘1’. Thus, the assessment enables the user or an administrator to take required actions based upon the assessment as one of safe-to-work and not safe-to-work.

FIG. 5B illustrates a flowchart showing a process for determining cough risk index in accordance with some embodiments of the present disclosure.

As illustrated in FIG. 5B, the method (506) comprises one or more blocks implemented by the processor (130) to compute cough risk index by using HMS (102). The method (506) may be described in the general context of computer executable instructions. Generally, computer executable instructions can include routines, programs, objects, components, data structures, procedures, modules, and functions, which perform specific functions or implement specific abstract data types.

The order in which the method (506) is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

At block (520), received cough sounds are analysed using the machine learning model (402). In one embodiment, the cough analysis module (136) analyses each of the retrieved plurality of cough sounds by using the machine learning model (402). The machine learning model (402) is trained with the training data (220) that comprises plurality of training datasets. The plurality of training datasets includes historical information related to cough sounds, cough pattern, pattern severity, underlying respiratory condition, cough severity score, dry cough count, wet cough count etc. of a group of healthy people and a group of unhealthy people suffering from respiratory diseases. In the process of training, the machine learning model (402) learns different patterns of cough sounds, one or more characteristics of cough sounds, correlation between plurality of cough sound characteristics etc.

At block (522), chough sound characteristics are determined for analysed cough sounds. In one embodiment, the cough analysis module (136) determines one or more cough sound characteristics for the received cough sounds based on the analysis of the machine learning model (402). Upon receiving the plurality of cough sounds, the machine learning model (402) determines one or more cough sound characteristics for each of the cough sounds by implementing learning from the training data (220). The determined one or more cough sound characteristics includes but not limited to underlying respiratory condition, cough pattern, pattern severity etc.

At block (524), total cough count is determined, and cough type is identified. In one embodiment, the cough analysis module (136) computes one or more cough sequences in each of the received plurality of cough sounds and number of coughs in each of the detected cough sequences. The cough analysis module (136) determines total count of coughs in each of the received plurality of cough sounds based on computed number of cough sequences and number of coughs in each of the cough sequences. Upon determining the total count of coughs, the cough analysis module (136) identifies type of cough as one of dry cough and wet cough for each cough in the cough sequences of the received plurality of cough sounds.

At block (526), the dry cough count and the wet cough count are computed based on total count of coughs and type of cough. In one embodiment, upon determining the total count of coughs and type of cough, the cough analysis module (136) determines total count of dry coughs and total count of wet cough in the received plurality of sough sounds.

At block (528), the dry cough severity and wet cough severity are computed based on the comparison of dry cough count and wet cough count with the respective pre-defined threshold values. In one embodiment, the risk assessment module (138) retrieves the first set and the second set of pre-defined threshold values for dry cough count and wet cough count from the threshold data (218) of the health data repository (106). The risk assessment module (138) further determines the dry cough severity and the wet cough severity based on the comparison of the determined dry cough count and wet cough count with the respective one the first set and the second set of pre-defined threshold values.

At block (530), the cough severity score is determined based on cough sound characteristics. In one embodiment, the risk assessment module (138) performs combined analysis of underlying respiratory condition, cough pattern, pattern severity, dry cough severity, and wet cough severity to determine the cough severity score of the plurality of received cough sounds. As illustrated in FIG. 6A, the risk assessment module (138) determines the cough severity score based on the pre-defined mapping of cough severity score with different values of underlying respiratory condition, cough pattern, pattern severity, dry cough severity, and wet cough severity.

At block (532), the cough risk index is determined based on cough sound characteristics and cough severity. In one embodiment, the risk assessment module (138) retrieves one of the first and the second pre-defined threshold values of the cough severity score based on the underlying respiratory condition and determines the cough risk index as one of ‘0’ and ‘1’ based on the comparison of the determined cough severity score with the one of the first and second pre-defined threshold values.

FIG. 5C illustrates a flowchart showing a process for generating alert in accordance with some embodiments of the present disclosure.

As illustrated in FIG. 5C, the method (550) comprises one or more blocks implemented by the processor (130) to compute cough risk index by using HMS (102). The method (550) may be described in the general context of computer executable instructions. Generally, computer executable instructions can include routines, programs, objects, components, data structures, procedures, modules, and functions, which perform specific functions or implement specific abstract data types.

The order in which the method (550) is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

At block (552), an alert is generated upon determining the user's health risk as not safe-to-work. In one embodiment, the risk assessment module (138) determines user's health risk as not safe-to-work when at least one of symptom risk index, cough risk index, temperature risk index, and oxygen saturation risk index is determined as ‘1’. Upon determining the user's health risk as not safe-to-work, the risk advisory module (232) generates an alert or notification that contains details of the associated health risk in one or more formats suitable for one or more transmission modes such as SMS (Short Message Services), email, app notification, automated phone calls etc.

At block (554), the generated alert is transmitted to the user displaying one or more risk indices. In one embodiment, the risk advisory module (232) transmits the generated alert or notification along with the symptom risk index, cough risk index, temperature risk index, oxygen saturation risk index, and final risk index to the user or one or more authorized persons related to the user.

At block (556), one or more recommended action items are generated for the user. In one embodiment, the risk advisory module (232) generates one or more actions items recommended for respective health risk such as symptom risk, cough risk, temperature risk, oxygen saturation risk based on the predefined actions items stored in the health data repository (106). The risk advisory module (232) further notifies the generated one or more recommended action items to the user or the one or more authorized person related to the user by using a suitable transmission mode.

At block (558), updates on one or more recommended action items are received to track progress. In one embodiment, the risk advisory module (232) receives updates from the user or one or more authorized person related to the user via app-based progress tracker, SMS (Short Message Services), automated confirmation call etc., wherein such updates are very crucial for avoiding the health risk of the user.

At block (560), the completion of the recommended action items is checked. In one embodiment, the risk advisory module (232) determines the progress on the recommended action items based on the received updates on such recommended action items. Upon determining incomplete status of the recommended action items, the risk advisory module (232) further transmits the pending recommended action items to the user or one or more authorized person related to the user. The risk advisory module (232) stops transmitting the recommended action items upon determination of completion of the previously issued recommended action items.

In one embodiment, the temperature reading, and oxygen saturation level of the user are regularly monitored over a period of time to generate more alerts. The data acquisition module (134) receives the temperature reading and oxygen saturation level of the user at a regular interval over a period of time and stores the received temperature reading and oxygen saturation level as temperature data (210) and SPO2 data (212) respectively in the health data repository (106). The risk trend analysis module (234) retrieves the stored temperature reading and oxygen saturation level from the temperature data (210) and SPO2 data (212) respectively and determines trends of temperature reading and oxygen saturation level of the user over the period of time with respect to the pre-defined threshold values. The risk trend analysis module (234) further predicts a surge of temperature reading or decline in oxygen saturation based on the determined trends and generates alerts to the user or one or more authorized person related to the used based on the prediction.

In another embodiment, cough severity score of the user is monitored over a period of time to generate alert based on cough severity in addition to temperature reading and oxygen saturation level. The data acquisition module (134) receives plurality of cough sounds of the user at a regular interval over a period of time and stores the received plurality of cough sounds as acoustic data (214). The cough analysis module (136) further stores the determined cough severity score of the user over a period of time as acoustic data (214) in the health data repository (106). The risk trend analysis module (234) retrieves the stored cough severity score from the acoustic data (214) and determines trends of cough severity score of the user over the period of time with respect to the first and second pre-defined threshold values. The risk trend analysis module (234) further predicts a surge in cough severity score based on the determined trends and generates alerts to the user or one or more authorized person related to the used based on the prediction.

As illustrated in FIG. 6C, the graphical representation comprises one or more elements to describe the trend analysis of wet cough count by the HMS (102). A graph (600) comprises a horizontal axis and a vertical axis, wherein days in a week i.e. Monday, Tuesday, . . . Sunday are plotted along the horizontal axis and range of wet cough count i.e. 0-20 is plotted along the vertical axis. The plotted lines (608, 606, 604, and 602) indicate variation in wet cough counts of the user at morning, noon, evening and night time respectively over period of one week and the plotted line (610) indicates the first pre-defined threshold value for wet cough count. The HMS (102) represents the graph (600) based on the processing of captured wet count of the user in a regular interval over a period of time. Therefore, the graph (600) depicts that the wet cough counts of the user increases at night-time and the wet cough counts at night exceeds the first pre-defined threshold value over the period of one week.

In analogous approach, FIG. 6D illustrates the graphical representation comprising one or more elements to describe the trend analysis of dry cough count by the HMS (102). A graph (650) comprises a horizontal axis and a vertical axis, wherein days in a week i.e. Monday, Tuesday, . . . , Sunday are plotted along the horizontal axis and range of dry cough count i.e. 0-20 is plotted along the vertical axis. The plotted lines (658, 656, 654, and 652) indicate variation in dry cough counts of the user at morning, noon, evening and night time respectively over period of one week and the plotted line (610) indicates the first pre-defined threshold value for dry cough count. The HMS (102) represents the graph (650) based on the captured dry count of the user in a regular interval over a period of time. Therefore, the graph (630) depicts that the dry cough counts of the user does not show any risk at throughout the day and the dry cough count further remains within the first pre-defined threshold value over the period of one week.

For example, a user, working in an organization, suffers from high fever and frequent cough and wishes to assess his/her safe-to-work conditions due to present clinical conditions such as fever. The user registers himself with the HMS (102) by using the user device (104) and providing necessary personal details such as contact information, height, weight etc. in the respective interface of the user application (116) as provided by the HMS (102). The user responds to a set of interactive questionnaires by providing appropriate answer in the user application (116). The HMS (102) receives the user response to the questionnaire and identifies one or more clinical symptom information based on the user response. The HMS (102) determines the symptom risk index (S) as ‘0’ based on the analysis of identified one or more clinical symptom information. The user application (116) further initiates the recording of cough sounds of the user by using the embedded acoustic sensor (118) in the user device (104). The HMS (102) receives the recorded cough sounds and determines the one or more cough sound characteristics based on the analysis of the cough sounds. The HMS (102) further determines a cough risk index (C) as ‘1’ based on the determined one or more cough sound characteristics. The HMS (102) receives the user's body temperature as determined by the temperature sensor (112-1) that is portably carried by the user and connected to the user device (104) via Bluetooth. Upon receiving the body temperature, the HMS (102) determines temperature risk index (T) as ‘1’ based on the received body temperature. The HMS (102) further receives the user's oxygen saturation information as measured by a SPO2 sensor (114-1) that is also portably carried by the user and connected to the user device (104) via Bluetooth and determines an oxygen saturation risk index (O) as ‘0’ based on the received oxygen saturation information. The HMS (102) generates a final risk index of the user as ‘1’ based on the determined symptom risk index (S), cough risk index (C), temperature risk index (T), and oxygen saturation risk index (O), wherein the final risk index is displayed in the user's personal device. Based on the final risk index, the HMS (102) predicts the user as not safe-to-work and provides the user with one or more recommended action items to mitigate risk associated with health.

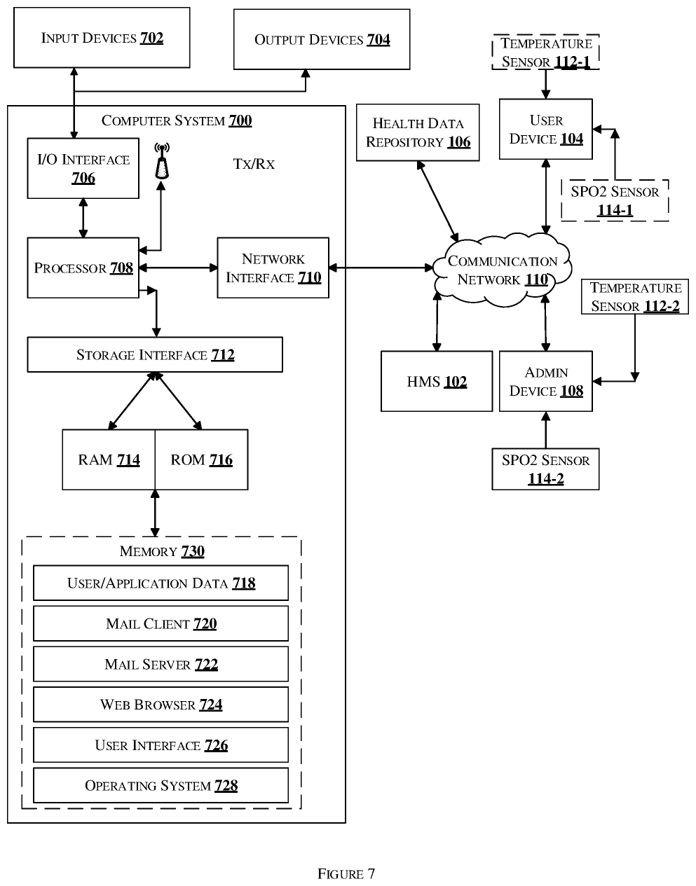

FIG. 7 illustrates a block diagram of an exemplary computer system for implementing embodiments consistent with the present disclosure.

In an embodiment, the computer system (700) may be health risk monitoring system (102), which is used for assessing health risk of a user by determining plurality of health parameters. The computer system (700) may include a central processing unit (“CPU” or “processor”) (708). The processor (708) may comprise at least one data processor for executing program components for executing user or system-generated business processes. The processor (708) may include specialized processing units such as integrated system (bus) controllers, memory management control units, floating point units, graphics processing units, digital signal processing units, etc.

The processor (708) may be disposed in communication with one or more input/output (I/O) devices (702 and 704) via I/O interface (706). The I/O interface (706) may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc.

Using the I/O interface (706), the computer system (700) may communicate with one or more I/O devices (702 and 704). In some implementations, the processor (708) may be disposed in communication with a communication network (110) via a network interface (710). The network interface (710) may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc. Using the network interface (710) and the communication network (110), the computer system (700) may be connected to the HMS (102), the user device (104), the health data repository (106), and the admin device (108).

The communication network (110) can be implemented as one of the several types of networks, such as intranet or any such wireless network interfaces. The communication network (110) may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network (110) may include a variety of network devices, including routers, bridges, servers, computing devices, storage devices, etc.

In some embodiments, the processor (708) may be disposed in communication with a memory (730) e.g., RAM (714), and ROM (716), etc. as shown in FIG. 7, via a storage interface (712). The storage interface (712) may connect to memory (730) including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory (730) may store a collection of program or database components, including, without limitation, user/application (718), an operating system (728), a web browser (724), a mail client (720), a mail server (722), a user interface (726), and the like. In some embodiments, computer system (700) may store user/application data (718), such as the data, variables, records, etc. as described in this invention. Such databases may be implemented as fault-tolerant, relational, scalable, secure databases such as Oracle or Sybase.

The operating system (728) may facilitate resource management and operation of the computer system (700). Examples of operating systems include, without limitation, Apple Macintosh™ OS X™, UNIX™, Unix-like system distributions (e.g., Berkeley Software Distribution (BSD), FreeBSD™, Net BSD™, Open BSD™, etc.), Linux distributions (e.g., Red Hat™, Ubuntu™, K-Ubuntu™, etc.), International Business Machines (IBM™) OS/2™, Microsoft Windows™ (XP™ Vista/7/8, etc.), Apple iOS™, Google Android™, Blackberry™ Operating System (OS), or the like. A user interface may facilitate display, execution, interaction, manipulation, or operation of program components through textual or graphical facilities. For example, user interfaces may provide computer interaction interface elements on a display system operatively connected to the computer system (700), such as cursors, icons, check boxes, menus, windows, widgets, etc. Graphical User Interfaces (GUIs) may be employed, including, without limitation, Apple™ Macintosh™ operating systems' Aqua™ IBM™ OS/2™, Microsoft™ Windows™ (e.g., Aero, Metro, etc.), Unix X-Windows™, web interface libraries (e.g., ActiveX, Java, JavaScript, AJAX, HTML, Adobe Flash, etc.), or the like.

The instant invention provides the technical solution to the technical problem of the existing health monitoring system by providing predictive analysis of a plurality of health parameters and integrating cough analysis as a significant parameter among the plurality of other health parameters such as body temperature, oxygen saturation, clinical symptoms etc. The integration of cough analysis in health risk determination results into more accurate prediction of health risk due to infectious disease like Covid-19, Tuberculosis etc. In case of any pandemic situation, the stepwise assessment feature of instant invention enables a user to perform health risk assessment at home without visiting any health centre, thereby preventing exposure of user to infections. The system eliminates health risk to employees in a workplace by automatically determining the health risk of each employee as safe-to-work or not safe-to-work based on the clinical symptoms and cough sound analysis of each employee. Further, the system can reduce spread of infection from any person susceptible to the infection by performing predictive analysis based on the trend of the health parameters. The predictive analysis can mitigate the impact of the infectious disease on the user by enabling the user to take preventive measures or alert the user with the upcoming dangerous health condition so as to take necessary medical treatment or other measures. Therefore, the system of the instant invention provides an automated, robust, highly scalable, dynamic and time-efficient platform to the one or more individuals or enterprises for performing health risk monitoring of an individual or a group of persons.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., are non-transitory. Examples include random access memory (RAM), read-only memory (ROM), volatile memory, non-volatile memory, hard drives, CD ROMs, DVDs, flash drives, disks, and any other known physical storage media.

Finally, the language used in the specification has been principally selected for readability and instructional purposes, and it may not have been selected to delineate or circumscribe the inventive subject matter. Accordingly, the disclosure of the embodiments of the disclosure is intended to be illustrative, but not limiting, of the scope of the disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

We claim:

1. A method for assessing health risk of a user, comprising:
receiving a plurality of user related information and a plurality of cough sounds as input from the user, wherein the user related information comprises at least user's clinical symptoms data;
determining a symptom risk index based on analysis of the received plurality of user related information;
computing a cough risk index based on determination of one or more cough sound characteristics obtained by processing the received plurality of cough sounds;
determining a temperature risk index and an oxygen saturation risk index based on analysis of measured temperature reading and oxygen saturation level of the user;
generating a final risk index based on the computed symptom risk index, cough risk index, temperature risk index, and oxygen saturation risk index; and
automatically assessing the health risk of the user as one of safe-to-work and not safe-to-work based on the final risk index.

2. The method as claimed in claim 1, wherein determining the symptom risk index comprising:
assigning a pre-defined weightage score to each of the clinical symptoms data, wherein the clinical symptoms includes basic health symptoms, cough related symptoms, external exposure information such as travel history, one or more medical conditions including diabetes, heart disease, lung disease, kidney problem, major operations;
computing a total weightage symptom score based on summation of pre-defined weightage score to each of the clinical symptoms data; and
determining the symptom risk index based on comparison of total weightage symptom score with a pre-defined threshold weightage symptom score.

3. The method as claimed in claim 1, wherein determining the temperature risk index and the oxygen saturation risk index comprising the step of comparing the body temperature reading and the oxygen saturation level of the user with respective predefined threshold values to determine the temperature risk index and the oxygen saturation risk index, wherein the pre-defined threshold values for temperature reading and oxygen saturation level are determined based on medically defined standard ranges.

4. The method as claimed in claim 1, wherein computing the cough risk index comprises steps of:
analysing the received plurality of cough sounds using a pre-trained machine learning model;
determining the one or more cough sound characteristics including underlying respiratory condition, cough pattern and pattern severity for each of the received plurality of cough sounds based on the analysis;
computing at least a dry cough severity and a wet cough severity for each of the plurality of cough sounds;
determining a cough severity score of the user based on a combined analysis of underlying respiratory condition, cough pattern, pattern severity, dry cough severity, and wet cough severity; and
determining the cough risk index based on comparison of the cough severity score with one of a first set and second set of pre-defined threshold values related to the cough severity score.

5. The method as claimed in claim 4, wherein computing at least the dry cough severity and the wet cough severity comprising:
computing one or more cough sequences in each of the received plurality of cough sounds, and number of coughs in each of the computed cough sequences;
determining total count of coughs in each of the received plurality of cough sounds based on computed number of cough sequences and number of coughs in each cough sequences;
identifying type of cough as one of dry cough and wet cough for each cough in the cough sequences of the received plurality of cough sounds;
determining at least one dry cough count and wet cough count based on total count of coughs and the type of cough for each of the plurality of cough sounds; and
computing at least dry cough severity and wet cough severity based on comparison of the dry cough count and wet cough count with one of the first set and the second set of pre-defined threshold values related to dry cough count and wet cough count respectively.

6. The method as claimed in claim 4, wherein the pre-trained machine learning model is generated by:
training the machine learning model with a set of training datasets comprising historic information of cough sound, and corresponding cough sound characteristics of a group of healthy people and group of unhealthy people suffering from respiratory diseases; and
determining a set of pre-defined threshold values related to each of the cough sound characteristics based on the historic information comprising:
determining the first set of pre-defined threshold values related to cough severity score, dry cough count, and wet cough count for the group of healthy people; and
determining the second set of pre-defined threshold values related to cough severity score, dry cough count, and wet cough count for the group of unhealthy people suffering from respiratory disease.

7. The method as claimed in claim 4, further comprising:
defining the individual threshold value for each of cough severity score, dry cough count, and wet cough count computed for each of the new user over a period of time;
dynamically updating the machine learning model upon receiving cough sound of one or more new users; and
dynamically updating the first set of pre-defined threshold values, the second set of pre-defined threshold values and the individual threshold values for each of the cough severity score, dry cough count, and wet cough count upon receiving cough sounds of one or more new users in the group by using the machine learning model.

8. The method as claimed in claim 1, further comprising:
generating an alert upon determining that the health risk of the user is not safe-to-work based on the final risk index;
transmitting the alert to the user and one or more authorized persons related to the user by displaying one or more of symptom risk index, cough risk index, temperature risk index, and oxygen saturation risk index;
generating one or more recommended action items to the user assessed as not safe-to-work; and
updating status relating to the one or more recommended action items from the user for tracking progress of one or more recommended action items.

9. The method as claimed in claim 8, further comprising:
monitoring the temperature reading, oxygen saturation level of the user at a regular interval over a period of time;
determining trends of temperature reading, oxygen saturation level of the user over a period of time with respect to the pre-defined threshold values;
predicting surge of temperature reading and decline of oxygen saturation level based upon the determined trends; and
generating alerts to the user based on the prediction.

10. The method as claimed in claim 9, further comprising:
monitoring the cough severity score of the user in a regular interval over a period of time;
determining trends of the cough severity score of the user over a period of time with respect to the pre-defined threshold values and individual threshold value;
predicting surge in cough severity score based upon the determined trends; and
generating alerts to the user based on the prediction.

11. A system for assessing health risk of the user, the system comprising:
a processor (130); and
a memory (132) communicatively coupled with the processor (130), wherein the memory (132) stores processor-executable instructions, which on execution, cause the processor (130) to:
receive a plurality of user related information and a plurality of cough sounds as input from the user via a user device coupled with the system, wherein the user related information comprises at least user's clinical symptoms data;
determine a symptom risk index based on analysis of the received plurality of user related information;
compute a cough risk index based on determination of one or more cough sound characteristics obtained by processing the received plurality of cough sounds;
determine a temperature risk index and an oxygen saturation risk index based on analysis of measured temperature reading and oxygen saturation level of the user;
generate a final risk index based on the computed symptom risk index, cough risk index, temperature risk index, and oxygen saturation risk index; and
automatically assess the health risk of the user as one of safe-to-work and not safe-to-work based on the final risk index.

12. The system as claimed in claim 11, wherein the processor (130) is configured to determine the symptom risk index, by:
assigning a pre-defined weightage score to each of the clinical symptoms data, wherein the clinical symptoms includes basic health symptoms, cough related symptoms, external exposure information such as travel history, one or more medical conditions including diabetes, heart disease, lung disease, kidney problem, major operations;
computing a total weightage symptom score based on summation of pre-defined weightage score to each of the clinical symptoms data; and
determining the symptom risk index based on comparison of total weightage symptom score with a pre-defined threshold weightage symptom score.

13. The system as claimed in claim 11, wherein the processor (130) is configured to determine the temperature risk index and the oxygen saturation risk index, by:
comparing the body temperature reading and the oxygen saturation level of the user with respective predefined threshold values to determine the temperature risk index and the oxygen saturation risk index, wherein the pre-defined threshold values for temperature reading and oxygen saturation level are determined based on medically defined standard ranges.

14. The system as claimed in claim 11, wherein the processor (130) is configured to compute the cough risk index, by:
analysing the received plurality of cough sounds using a pre-trained machine learning model;
determining the one or more cough sound characteristics including underlying respiratory condition, cough pattern and pattern severity for each of the received plurality of cough sounds based on the analysis;
computing at least a dry cough severity and a wet cough severity for each of the plurality of cough sounds;
determining a cough severity score of the user based on a combined analysis of underlying respiratory condition, cough pattern, pattern severity, dry cough severity, and wet cough severity; and
determining the cough risk index based on comparison of the cough severity score with one of a first set and a second set of pre-defined threshold values related to the cough severity score.

15. The system as claimed in claim 14, wherein the processor (130) is configured to compute at least the dry cough severity and the wet cough severity, by:
computing one or more cough sequences in each of the received plurality of cough sounds, and number of coughs in each of the computed cough sequences;
determining total count of coughs in each of the received plurality of cough sounds based on computed number of cough sequences and number of coughs in each cough sequences;
identifying type of cough as one of dry cough and wet cough for each cough in the cough sequences of the received plurality of cough sounds;
determining at least one dry cough count and wet cough count based on total count of coughs and the type of cough for each of the plurality of cough sounds; and
computing at least dry cough severity and wet cough severity based on comparison of the dry cough count and wet cough count with one of the first set and the second set of pre-defined threshold values related to dry cough count and wet cough count respectively.

16. The system as claimed in claim 14, wherein the pre-trained machine learning model is generated by:
training the machine learning model with a set of training datasets comprising historic information of cough sound, and corresponding cough sound characteristics of a group of healthy people and group of unhealthy people suffering from respiratory diseases; and
determining a set of pre-defined threshold values related to each of the cough sound characteristics based on the historic information comprising:
determining the first set of pre-defined threshold values related to cough severity score, dry cough count, and wet cough count for the group of healthy people; and
determining the second set of pre-defined threshold values related to cough severity score, dry cough count, and wet cough count for the group of unhealthy people suffering from respiratory disease.

17. The system as claimed in claim 14, wherein the processor (130) is further configured to:
define the individual threshold value for each of cough severity score, dry cough count, and wet cough count computed for each of the new user over a period of time;
dynamically update the machine learning model upon receiving cough sound of one or more new users; and
dynamically update the first set of pre-defined threshold values, the second set of predefined threshold values and the individual threshold values for each of the cough severity score, dry cough count, and wet cough count upon receiving cough sounds of one or more new users in the group by using the machine learning model.

18. The system as claimed in claim 11, wherein the processor (130) is configured to:
generate an alert upon determining that the health risk of the user is not safe-to-work based on the final risk index;
transmit the alert to the user and one or more authorized persons related to the user by displaying one or more of symptom risk index, cough risk index, temperature risk index, and oxygen saturation risk index;
generate one or more recommended action items to the user assessed as not safe-to-work; and
update status relating to the one or more recommended action items from the user for tracking progress of one or more recommended action items.

19. The system as claimed in claim 18, wherein the processor (130) is further configured to:
monitor the temperature reading, oxygen saturation level of the user at a regular interval over a period of time;
determine trends of temperature reading, oxygen saturation level of the user over a period of time with respect to the pre-defined threshold values;
predict surge of temperature reading and decline of oxygen saturation level based upon the determined trends; and
generate alerts to the user based on the prediction.

20. The system as claimed in claim 19, wherein the processor (130) is further configured to:
monitor the cough severity score of the user in a regular interval over a period of time;
determine trends of the cough severity score of the user over a period of time with respect to the pre-defined threshold values and individual threshold value;
predict surge in cough severity score based upon the determined trends; and
generate alerts to the user based on the prediction.
